(19) United States
(12) Patent Application Publication
(10) Pub. No.: US 2008/0076718 A1
(43) Pub. Date: Mar. 27, 2008

(54) Novel Proteasome Modulators

(76) Inventors: Michele Reboud-Ravaux, Paris (FR); Elise Bernard, Chavanne (FR); David Papapostolou, Fontenay Sous Bois (FR); Regis Vanderesse, Sexey Les Bois (FR)

Correspondence Address: MORGAN LEWIS & BOCKIUS LLP, 1111 PENNSYLVANIA AVENUE NW, WASHINGTON, DC, 20004, US

(21) Appl. No.: 10/583,282

(22) PCT Filed: Dec. 17, 2004
(86) PCT No.: PCT/FR04/03283

§ 371 Date: Jun. 27, 2007

(30) Foreign Application Priority Data
Dec. 18, 2003 (FR) 03 14958

(51) Int. Cl. C07K 7/06; A61K 38/07; A61K 38/08; A61K 8/64; A61P 25/00; A61P 31/18; A61P 35/00; A61P 9/00; C07K 5/10

(52) U.S. Cl. 514/15; 514/16; 514/17; 514/18; 530/328; 530/329; 530/330

(57) ABSTRACT

The invention relates to novel proteasome activity modulating molecules which are used in pharmaceutical and cosmetic compositions for preventing and/or treating proteasome-induced pathologies and disorders.

The present invention relates to novel molecules and to the use thereof for modulating proteasome activity. It also relates to the pharmaceutical and cosmetic compositions containing them and to the use of these molecules for preventing and/or treating proteasome-related pathologies and disorders.

The proteasome is an essential proteolytic enzyme of the cytoplasm and of the nucleus of eukaryotic cells. It is involved in the degradation of most intracellular proteins and participates in the transformation of the antigens presented by most MHC-1 molecules.

At least five types of proteolytic activities have been identified, including three main ones: a chymotrypsin-like activity (CT-L), a trypsin-like activity (T-L) and a post-acid peptidase activity. The catalytic site of post-acid peptidase type preferentially cleaves peptide sequences comprising a glutamic acid in position P1; the trypsin-like catalytic site preferentially but not exclusively cleaves peptide sequences comprising a basic amino acid (arginine, lysine) in position P1; the chymotrypsin-like catalytic site preferentially but not exclusively cleaves peptide sequences comprising a hydrophobic amino acid, such as leucine, in position P1.

The structure of the proteasome is that of a 26S protein complex (2.4 MDa) comprising a catalytically active complex called 20S, the activity of which is regulated by complex regulators.

The proteasome hydrolyzes proteins to fragments of 3 to 25 residues with an average of 7 to 8 residues.

The catalytic particle of the proteasome, 20S, can be in two distinct states, one being activated and the other being nonactivated.

The proteasome is an element essential to intracellular proteolysis, whether or not it is ubiquitin-dependent (Eytan et al., Proc. Natl. Acad. Sci. USA 86:7751-7755 (1989); Reichsteiner et al., J. Biol. Chem. 268:6065-6068 (1993)). These mechanisms are involved in the degradation of cyclins and of other short-lifespan and long-lifespan proteins. Oncogenes (Glotzer et al., Nature 349:132-138 (1991); Ciechanover et al., Proc. Natl. Acad. Sci. USA 88, 139-143 (1991)) and ornithine decarboxylase (Murakami et al., Nature, 360:597-599 (1992)) constitute examples of degraded proteins. These data strongly suggest that the proteasome plays an important role in the regulation of cell growth and in mitosis.

The proteasome also plays a key role in the presentation of antigenic peptides to the cells of the immune system, and therefore in the surveillance directed against viruses and cancer (Brown et al., Nature, 355:355-360 (1991)).

The role played by the proteasome in protein degradation suggests that inhibition of said proteasome may make it possible to act on pathologies such as cancer, autoimmune diseases, AIDS, inflammatory diseases, cardiac diseases, transplant rejection, or amyotrophy (M. Reboud-Ravaux, Progress in Molecular and Subcellular Biology, vol. 29, Springer Verlag, 2002, p. 109-125; Kisselev et al., Chemistry & Biology, 8, 739-758 (2001)).

Moreover, it is known that activation of the proteasome should make it possible to act on the mechanisms of intracellular proteolysis in the direction of an acceleration of these mechanisms, which may be desired, for example, when an accumulation of oxidized proteins is observed. In this context, a proteasome-activating molecule should make it possible to eliminate the oxidized proteins and should constitute a treatment and/or a method for inhibiting the appearance of the signs of aging, in particular of skin aging. Proteasome-activating molecules have been described in particular by: Kisselev et al., J. Biol. Chem., 277, 22260-22270 (2002); Wilk et al., Mol. Biol. Rep., 24, 119-124 (1997); Ruiz De Mena et al., Biochem. J., 296, 93-97 (1993); Arribas et al., J. Biol. Chem., 265, 13969-13973 (1990).

Protein accumulation is also observed in the context of Alzheimer's disease and in Parkinson's disease. Proteasome activation could make it possible to activate the protein degradation process in the treatment of these pathologies. Compounds of this type are described in documents U.S. Pat. No. 5,847,076 and JP-2002029996.

A proteasome inhibitor already exists on the market: Velcade® is used for the treatment of multiple myeloma. Velcade® binds covalently to the active sites of the proteasome and thus blocks their activity. It thus prevents the proteasome from carrying out protein degradation and blocks in particular the apoptosis and cell death process (Richardson et al., Cancer Control, 10, 361-366 (2003)).

However, this mechanism of action, which is extremely effective, is also found to be toxic for the organism and results in considerable side effects. The problem is therefore that of finding proteasome inhibitors which are less drastic in terms of their mechanism of action.

The difficulty in defining proteasome inhibitors is all the greater since the proteasome shows mediocre specificity in the choice of its substrates and in the cleavage scheme that it adopts.

One of the problems that the invention is intended to solve was that of the development of molecules that bind noncovalently to the active sites of the proteasome and/or to the regulatory sites of the proteasome.

The document Bioorganic and Medicinal Chemistry, 11 (2003), 4881-4889 describes pseudopeptides derived from the sequence Ac-Leu-Leu-Norleucinal. These compounds are potential proteasome inhibitors. However, their activity on the proteasome is not quantified.

It has also been sought to develop small molecules whose synthesis is simple and reproducible in order to be industrializable. It has also been desired to obtain molecules which are stable, including for oral administration.

The document Papapostolou et al., BBRC, 295 (2002) 1090-1095 describes small peptides (5 to 6 amino acids) which bind noncovalently to the proteasome and which have a modulatory activity (activating activity for some, inhibitory activity for others) on the functions of the proteasome.

However, the affinity of these molecules for their target can also be improved and their stability under conditions for administration to a human organism leave a lot to be desired.

The inventors therefore set themselves the objectives of designing and synthesizing novel molecules which do not have the drawbacks of the molecules of the prior art.

This objective was achieved through the molecules of the invention which correspond to general formula (I) below, and the pharmaceutically acceptable salts thereof:

(X₀)_{X0}—(X₁)_{X1}—(X₂)_{X2}—X₃—(X₄)_{X4}—X₅—X₆—(X₇)_{X7}—(X₈)_{X8}—(X₉)_{X9} (I)

in which x₀, x₁, x₂, x₄, x₇, x₈ and x₉ each represent, independently, an integer equal to 0 or to 1;

x₀ epresents a group chosen from those corresponding to formula (II):

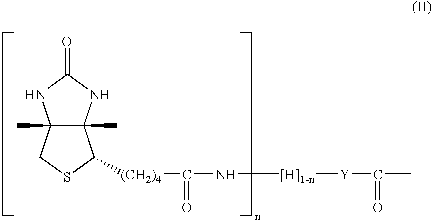

in which Y represents a saturated or unsaturated, linear, branched or cyclic C₁-C₂₄ alkyl group, n represents an integer chosen from 0 and 1.

Depending on the case:

n=1 and X₀ represents a biotinyl group grafted onto an aminoacyl chain;

n=0 and X₀ represents an acyl chain HY—CO-;

X₁ and X₃ each represent a natural or synthetic amino acid in the L or D configuration, each comprising at least one hydroxyl function on its side chain. X₁ and X₃, which may be identical or different, can be chosen, for example, from threonine and serine;

### X₂ represents a natural or synthetic amino acid in the L or D configuration which can be chosen from those comprising an alkyl side chain, such as, for example, valine, leucine or isoleucine;

X₄ represents a natural or synthetic amino acid in the L or D configuration which can be chosen from those comprising an aromatic side chain, such as, for example, phenylalanine, tryptophan or tyrosine; X₄ can also be an aromatic amino acid comprising a photoactivatable reactional group such as para-benzoylphenylalanine;
X₅ represents an amino acid in the L or D configuration selected from: positively charged amino acids such as lysine, arginine or histidine; negatively charged amino acids such as aspartic acid or glutamic acid; amino acids bearing an amide function, such as asparagine or glutamine;
X₆ represents an amino acid in the L or D configuration which can be chosen from tyrosine, phenylalanine, leucine, isoleucine and alanine; X₆ can also be an aromatic amino acid comprising a photoactivatable reactional group such as para-benzoylphenylalanine; X₆ can also be lysine;

### X₇ represents an amino acid in the L or D configuration which can be chosen from glycine, alanine, leucine, valine, asparagine and arginine;

### X₈ represents an amino acid in the L or D configuration which can be chosen from proline, valine, isoleucine and aspartic acid;

### X₉ represents an amino acid in the L or D configuration which can be chosen from serine, alanine, lysine, arginine and tryptophan;

the bond between two successive amino acids Xᵢ−Xᵢ₊₁, denoted qᵢ₋ᵢ₊₁, i=1, . . . 8, can be a peptide bond

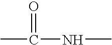

or a pseudopeptide bond chosen in particular from the following list:

| | |
|---|---|
| ester | CO—O |
| thioester | CO—S |
| keto methylene | CO—CH₂ |
| N-methylamide | CO—N(Me) |
| inverse amide | NH—CO |
| Z/E vinylene | CH═CH |
| ethylene | CH₂—CH₂ |
| methylenethio | CH₂—S |
| methyleneoxy | CH₂—O |
| thioamide | CS—NH |
| methyleneamino | CH₂—NH |
| keto methyleneamino | CO—CH₂—NH |
| hydrazino | CO—NH—NH |
| carbonylhydrazone | CO—NH—N═ |
| N-amino | CO—N(NH₂) |

the amino acids stated above Xᵢ, i=1, . . . 9, being capable of comprising a modification of their α-carbon, denoted Cᵢ, i=1, . . . 9, and bearing the side chain R of the amino acid, which modification consisting of the replacement of:

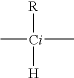

with a group chosen from:

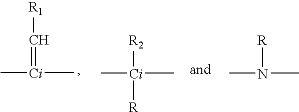

the groups R and CH—R₁ representing the side chain of the amino acid and R₂ representing a C₁-C₆ alkyl group; optionally, R-R₂ can constitute a ring,
the pseudopeptides of the invention also corresponding to the following conditions:
x₀ is equal to 1 or
one of the bonds qᵢ₋ᵢ₊₁, i=1, . . . 8, is a pseudopeptide bond or
one of the Cᵢ, i=1, . . . 9, comprises one of the modifications stated above.

In fact, as is illustrated in the experimental section, the molecules of formula (I), which comprise at least one nonpeptide group, have in common the property of binding noncovalently to the active sites and/or to the regulatory sites of the proteasome. In particular, they have the property of binding to the active sites and/or to the regulatory sites of the CT-L (chymotrypsin-like) activity of the proteasome.

Some of these molecules have a proteasome-inhibiting activity, others are proteasome-activators. Some molecules, comprising a para-benzoylphenylalanine photoactivatable group, can, through the application of a photochemical treatment, bind covalently to the proteasome.

It has been noted that, in tests carried out in vitro, the molecules of the invention have a greater affinity for the proteasome than the molecules of the prior art described in Papapostolou et al., BBRC, 295 (2002) 1090-1095, which have a strictly peptide structure.

Furthermore, their not strictly peptide nature (the presence of nonpeptide bond(s) and/or of certain modified amino acids) makes it possible to envisage a reduced effectiveness of proteases on the degradation of these molecules and therefore better resistance to proteolysis under in vivo administration conditions.

In addition to the pseudopeptide characteristics stated above, the amino acids used for the preparation of the molecules of formula (I) can be natural amino acids, in the form of the L enantiomer. However, the use of the D analogs thereof or the β-amino, γ-amino or ω-amino analogs thereof can be envisioned.

The molecules of the invention comprise at least one of the following characteristics:
biotinyl or acyl chain at the N-terminal end,
or modified peptide bond,
or presence of an amino acid comprising a modified α-carbon, each of these modifications consisting of a variant with respect to a simple peptide chain:

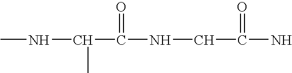

However, the molecules of the invention can comprise more than one modification with respect to a simple peptide chain, such as, for example:
an acyl group at the N-terminal end and one or more pseudopeptide bonds,
a biotinyl group at the N-terminal end and a para-benzoylphenylalanine group in the peptide chain,
a pseudopeptide bond and an amino acid comprising a modified α-carbon,
an N-terminal acyl group and a β- or γ-amino acid.

When x₀=1, the acyl chain —Y—CO— may be linear, branched or cyclic, and saturated or unsaturated. Preferably it is a linear chain which is represented by the formula —CₚH₂ₚ—CO—, p being an integer ranging from 1 to 23.

Preferably, at least one of the integers x₀, x₁, x₂, x₄, x₇, x₈ and x₉ is equal to 1.

Among the molecules corresponding to formula (I), those comprising 4 to 8 amino acids, preferably 5 to 7 amino acids, even more preferably those comprising 6 amino acids, are preferred.

In the case where x₀=1:
when n=1, preferably Y contains 1 to 8 carbon atoms, for example Y represents —CₚH₂ₚ— and p can be 1, 2, 3, 4, 5, 6, 7 or 8,

when n=0, preferably Y contains from 5 to 23 carbon atoms, for example Y represents —CₚH₂ₚ— and p can be an integer ranging from 5 to 23.

Preferably, at least one of X₁ and of X₃ represents threonine. Even more preferably, X₁ and X₃ both represent threonine.

Preferably, X₂ is chosen from isoleucine and valine.

Preferably, X₄ is chosen from phenylalanine, tyrosine and para-benzoylphenylalanine.

Preferably, at least 2 of the integers x₀, x₁, x₂, x₄, x₇, x₈ and x₉ are equal to 1, even more preferably at least 3 of these integers are equal to 1.

Among the molecules corresponding to formula (I), a preferred sequence is that corresponding to formula (Ia):

X₀−X₁−X₂−X₃−X₄−X₅−X₆ (Ia)

in which X₀, X₁, X₂, X₃, X₄, X₅ and X₆ have the same definition as above, the bonds qᵢ, ᵢ₊₁, between the amino acids Xᵢ and Xᵢ₊₁, i=1, . . . 5, being peptide or pseudopeptide bonds.

According to a first preferred variant of the molecule (Ia), X₀ represents:

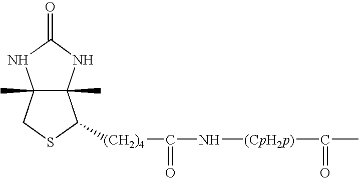

with p ranging from 1 to 8, preferably from 2 to 6, and X₄ represents a para-benzoylphenylalanine group.

According to a second preferred variant of the molecule (Ia), X₀ represents an acyl group:

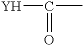

in which Y represents a C₃-C₂₃ alkyl group.

Even more preferably, X₀ represents a group:

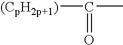

with p ranging from 3 to 23, preferably from 5 to 19.

Among the molecules corresponding to formula (I), another preferred sequence is that corresponding to formula (Ib):

X₃—X₅—X₆—X₇—X₈—X₉ (Ib)

in which X₃, X₅, X₆, X₇, X₈ and X₉ have the same definition as above,
at least one of the bonds between two successive amino acids being a pseudopeptide bond or
one of the α-carbons of one of the amino acids being a modified α-carbon.

According to the invention, the term “salts” relates both to the amine salts of a carboxyl function of the peptide chain and to the acid addition salts with an amine group of this same polypeptide chain. The salts of a carboxyl function can be formed with an inorganic or organic base. The inorganic salts include, for example, alkali metal salts such as sodium salts, potassium salts and lithium salts; alkaline earth metal salts such as, for example, calcium salts, barium salts and magnesium salts; ammonium salts, ferrous salts, ferric salts, zinc salts, manganese salts, aluminum salts, magnesium salts. The salts with organic amines include those formed, for example, with trimethylamine, triethylamine, tri(n-propyl)amine, dicyclohexylamine, triethanolamine, arginine, lysine, histidine, ethylenediamine, glucosamine, methylglucamine, purines, piperazines, piperidines, caffeine and procaine.

The acid addition salts include, for example, salts with inorganic acids such as, for example, hydrochloric acid, hydrobromic acid, sulfuric acid, phosphoric acid or nitric acid; salts with inorganic acids such as, for example, acetic acid, trifluoroacetic acid, oxalic acid, tartaric acid, succinic acid, maleic acid, fumaric acid, gluconic acid, citric acid, malic acid, ascorbic acid or benzoic acid.

Among the preferred molecules of the invention, mentioned may be made of:
CH₃—(CₙH₂ₙ)—CO-TVTYDY with n=4,6,8,10,12,14,16,18
CH₃—(CₙH₂ₙ)—CO-TISYDY with n=4,6,8,10,12,14,16,18
CH₃—(CₙH₂ₙ)—CO-TVSYKF with n=4,6,8,10,12,14,16,18
CH₃—(CₙH₂ₙ)—CO-TITFDY with n=4,6,8,10,12,14,16,18
CH₃—(CₙH₂ₙ)—CO-TITYKF with n=4,6,8,10,12,14,16,18
CH₃—(CₙH₂ₙ)—CO-TITYEY with n=4,6,8,10,12,14,16,18
CH₃—(CₙH₂ₙ)—CO-TITYDF with n=4,6,8,10,12,14,16,18
CH₃—(CₙH₂ₙ)—CO-TVTYKL with n=4,6,8,10,12,14,16,18
CH₃—(CₙH₂ₙ)—CO-TVTYKY with n=4,6,8,10,12,14,16,18
CH₃—(CₙH₂ₙ)—CO-TVTFKF with n=4,6,8,10,12,14,16,18
CH₃—(CₙH₂ₙ)—CO-TITYDL with n=4,6,8,10,12,14,16,18
CH₃—(CₙH₂ₙ)—CO-TITFDY with n=4,6,8,10,12,14,16,18
CH₃—(CₙH₂ₙ)—CO-TVTFKF with n=4,6,8,10,12,14,16,18
CH₃—(CₙH₂ₙ)—CO-TVTYKF with n=4,6,8,10,12,14,16,18
Biot-Ava-TVT-Bpa-KF
Biot-Ava-TVT-Bpa-KY
Biot-Ava-TVT-Bpa-KL
Biot-Ava-TVT-Bpa-DF
Biot-Ava-TVT-Bpa-DY
Biot-Ava-TVT-Bpa-DL
Biot-Ava-TIT-Bpa-KF
Biot-Ava-TIT-Bpa-KY
Biot-Ava-TIT-Bpa-KL
Biot-Ava-TIT-Bpa-DF
Biot-Ava-TIT-Bpa-DY
Biot-Ava-TIT-Bpa-DL
Biot-Ava-TVT-Bpa-EF
Biot-Ava-TVT-Bpa-EY
Biot-Ava-TVT-Bpa-EL
Biot-Ava-TIT-Bpa-EF
Biot-Ava-TIT-Bpa-EY
Biot-Ava-TIT-Bpa-EL
Biot-Ava-TVT-Bpa-NF
Biot-Ava-TVT-Bpa-NY
Biot-Ava-TVT-Bpa-NL
Biot-Ava-TIT-Bpa-NF
Biot-Ava-TIT-Bpa-NY
Biot-Ava-TIT-Bpa-NL

TNL*GPS, or else SEK*RVW, TRA*LVR, SNL*NDA and THI*VIK, in which * represents:
a bond chosen from ester, thioester, keto methylene, keto methyleneamino, N-methylamide, inverse amide, Z/E vinylene, ethylene, methylenethio, methyleneoxy, thioamide, methyleneamide, hydrazino, carbonylhydrazone and N-amino bonds, or
the presence of an aza-amino acid as a substitution for one of the amino acids adjacent to *.

Biot represents a biotinyl group, Ava represents a δ-aminovaleric acid, Bpa represents a para-benzoylphenylalanine group.

According to the invention, it can also be envisioned that the molecules described above are coupled on their C-terminal end and/or when this is possible, on their N-terminal end, with another molecule which promotes the bioavailability of the molecule of the invention. To this effect, mention may in particular be made of the peptides which promote penetration into the cell and which are described in particular in: ROJA et al., Nat. Biotechnol., 16, 370-375 (1998); FUTAKI et al., J. Biol. Chem., 276, 5836-5840 (2001); MORRIS et al., Nat. Biotechnol., 19, 1173-1176 (2001). Mention may also be made of the product called penetratin and the peptide vectors sold by the company Diatos.

The molecules of the invention can be prepared according to techniques well known to those skilled in the art, such as peptide synthesis and pseudopeptide synthesis. These synthesis techniques are illustrated in the experimental section. For the synthesis of pseudopeptides, reference may, for example, be made to: SPATOLA, Vega Data, Vol. 1, issue 3 (1983); SPATOLA, Chemistry and Biochemistry of Amino Acids Peptides and Proteins, Weinstein, ed., Marcel Dekker, New York, p. 267 (1983), MORLEY, J.-S., Trends Pharm. Sci., 463-468 (1980); HUDSON et al., Int. J. Pept. Prot. Res. 14, 177-185 (1979); SPATOLA et al., Life Sci., 38, 1243-1249 (1986); Hann, J. Chem. Soc. Perkin Trans. I 307-314 (1982); ALMQUIST et al., J. Med. Chem., 23, 1392-1398 (1980); JENNINGS-WHITE et al., EP-45665; HOLLADAY et al., Tetrahedron Lett. 24, 4401-4404 (1983), HRUBY et al., Life Sci. 31, 189-199 (1982).

A modified peptide according to the invention can also be obtained by expression of a peptide from a recombinant nucleic acid molecule and then modification (grafting of a para-benzoyl group onto a phenylalanine residue, grafting of a biotinylaminoacyl group, or of an acyl group).

The molecules of the invention can be used for modulating proteasome activity; these uses constitute another subject of the invention.

A subject of the invention is in particular the use of a molecule described above, for preparing a medicinal product for use in the prevention and/or treatment of a pathology involving the proteasome, and in particular its chymotrypsin-like (CT-L) activity.

Some of these molecules have proteasome activity-inhibiting properties, and, in this respect, they can be used for preparing a medicinal product for use in the prevention and/or treatment of a pathology selected from: cancers involving hematological tumors, such as multiple myeloma, leukemias, lymphomas, sarcomas: RICHARSON et al., Cancer Control, 10, 361-366 (2003); ADAMS, Drugs Discovery Today, 8, 307-311; or solid spleen tumors, breast tumors, colon tumors, kidney tumors, ear/nose/throat tract tumors, lung tumors, ovarian tumors, prostate tumors, pancreatic tumors, skin tumors: LENZ, Cancer Treatment Reviews, 29, 41-48 (2003); inflammatory diseases such as, for example, Crohn's disease and asthma: ELLIOT et al., J. Allergy Clin. Immunol. 104, 294-300 (1999); ELLIOT et al., Journal of Molecular Medicine, 81, 235-245 (2003); amyotrophy: LECKER et al., J. Nutr. 129, 2275-2375 (1999); AIDS: SCHUBERT, Proc. Natl. Acad. Sci. USA, 97, 1357-1362 (2000); autoimmune diseases such as, for example, rheumatoid arthritis and acute disseminated lupus erythematosus; Schwartz et al., J. Immunol. 164, 6114-6157 (2000); cardiac pathologies such as, for example, myocarditis and the consequences of ischemic processes, whether at the myocardial, cerebral or pulmonary level: CAMPBELL et al., J. Mol. Cell. Cardiol. 31, 467-476; cerebral strokes: ZHANG et al., Curr. Drug Targets Inflamm. Allergy 1, 151-156 (2002), DI NAPOLI et al., Current Opinion Invest. Drugs, 4, 303-341 (2003), allograft rejection; traumas, burns, corneal regeneration: STRAMER et al., Invest. Ophthalmol. Vis. Sci. 42, 1698-1706 (2001).

Some of these molecules have a proteasome action-stimulating activity and, in this respect, they can be used for preparing a medicinal product for use in the prevention or treatment of certain pathologies related to aging, such as, for example, Alzheimer's disease: TSUJI and SHIMOHAMA in M. Reboud-Ravaux, Progress in Molecular and Subcellular Biology, vol. 29, Springer Verlag, 2002, p. 42-60, and Parkinson's disease: SIDELL et al., J. Neur. Chem., 79, 510-521 (2001).

The proteasome action-stimulating molecules can also be used in cosmetics or in dermatology, for preparing compositions intended to delay and/or treat the effects of chronological skin aging or actinic skin aging (photoaging): FISHER et al., Photochem. Photobiol. 69, 154-157 (1999). Oxidized proteins accumulate in the old fibroblasts of the skin, while the proteasome, responsible for the degradation of the oxidized proteins, experiences a decrease in its activity: GRUNE, Hautartz, 54, 818-821 (2003); LY et al., Science, 287, 2486-2492 (2000). A subject of the invention is in particular a cosmetic process for preventing or treating the appearance of the effects of physiological and/or actinic skin aging, comprising the application of a molecule according to the invention, in a cosmetically acceptable carrier. Among the symptoms of skin aging, mention may in particular be made of the appearance of wrinkles, a dull complexion, relaxation of the skin, and the loss of elasticity.

The molecules of the invention can be used alone or in combination with one or more other active ingredients, both in the therapeutic field (anticancer treatment, anti-AIDS polytherapy, etc.) and in the cosmetics field. They can also be used jointly with a radiotherapy treatment.

The molecules of the invention can also be used for preparing a medicinal product for use in the radiosensitization of a tumor.

A subject of the invention is also a medicinal product comprising molecules of the invention in a pharmaceutically acceptable carrier.

The choice of the carrier and of the adjuvants will be guided by the method of administration that will be adjusted according to the type of pathology to be treated. Oral or parenteral administration can be envisioned.

The amount of molecule of formula (I) to be administered to humans, or optionally to animals, depends on the activity specific to this molecule, which activity can be measured by means which will be disclosed in the examples. It also depends on the degree of seriousness of the pathology to be treated.

A subject of the invention is also a cosmetic and/or dermatological composition comprising a molecule of the invention in a cosmetically and/or dermatologically acceptable carrier. Such a carrier may, for example, be a cream, a lotion, a milk, an ointment or a shampoo.

### EXPERIMENTAL SECTION

### A—Synthesis of Molecules

### 1—Lipopeptides

17 lipopeptides were synthesized, their structure is given in Table I:

TABLE I
Sequences synthesized

| Sequences | TITFDY | TVTFKF | TVTYKF |
|---|---|---|---|
| Aliphatic chain | CH₃—(CH₂)₄—CO— | CH₃—(CH₂)₄—CO— | CH₃—(CH₂)₄—CO— |
| | CH₃—(CH₂)₆—CO— | CH₃—(CH₂)₆—CO— | CH₃—(CH₂)₆—CO— |
| | CH₃—(CH₂)₈—CO— | CH₃—(CH₂)₈—CO— | CH₃—(CH₂)₈—CO— |
| | | CH₃—(CH₂)₁₀—CO— | CH₃—(CH₂)₁₀—CO— |
| | | CH₃—(CH₂)₁₂—CO— | CH₃—(CH₂)₁₂—CO— |
| | | CH₃—(CH₂)₁₄—CO— | CH₃—(CH₂)₁₄—CO— |
| | | CH₃—(CH₂)₁₆—CO— | CH₃—(CH₂)₁₆—CO— |

The lipopeptides are synthesized on a semiautomatic synthesizer (CNRS, IBMC, Strasbourg, France) (1. Neimark, J., and Briand, J. P. (1993) Pept. Res. 6, 219-228) using Fmoc-Leu(tBu)-Wang PS, Fmoc-Lys(Boc)-Wang PS and Fmoc-Tyr(tBu)-Wang PS resins (Senn Chemicals International (Dielsdorf, Switzerland)). The strategy used is a conventional Fmoc/tBu protocol. The peptide chain elongation is carried out by successive coupling and deprotection of the Fmoc-amino acids (3 eq. with respect to the substitution of the resin). The amino acids used (Neosystem (Strasbourg, France) or Senn Chemicals International (Gentilly, France)) are: Fmoc-Thr(tBu-OH, Fmoc-Tyr(tBu)-OH, Fmoc-Ser(tBu)-OH, Fmoc-Asp(OtBu)-OH, Fmoc-Gln(OtBu)-OH and Fmoc-Lys(Boc)-OH. The coupling catalysts are 2-(1H-benzotriazol-1-yl)-1,1,3,3-tetramethyluronium tetrafluoroborate (TBTU), (3 eq.), 1-hydroxybenzotriazole (HOBt) (3 eq.) and diisopropylethylamine (DIEA) (9 eq.) in N,N-dimethylformamide (DMF).

The progress of each step is controlled by means of a colorimetric assay using 2,4,6-trinitrobenzenesulfonic acid. The N-terminal deprotection of the Fmoc group is carried out with a 20% solution of piperidine in DMF.

The lipid chain is coupled using acid chlorides (3 eq.) in the presence of DIEA (9 eq.).

The peptides are cleaved from the resin for 2 hours with a mixture of 10 ml of TFA, 0.750 g of phenol, 0.25 ml of EDT, 0.5 ml of thioanisole and 0.5 ml of deionized water. This mixture is initially added to the resin-peptide at 0° C., but the cleavage is carried out at ambient temperature. The peptides precipitate through the addition of ice-cold Et₂O and the resin is filtered off. The peptide that has remained on the sintered glass is dissolved over a round-bottom flask full of ice-cold Et₂O using TFA. It is then concentrated and lyophilized.

The peptides are purified by high performance liquid chromatography (HPLC) carried out on a Hitachi-Merck system equipped with an L6200 pump coupled to a Jasco 875 UV detector. The preparative column used is a Macherey-Nagel Nucleosil 300-7 C4 column (250×10 mm i.d.). The eluant is composed of a solution A of 0.1% by volume of TFA (sequencing grade, Sigma) in Ultrapure water and of a solution B of 0.08% of TFA and of 20% of water in acetonitrile (Carlo Erba). The peptide is eluted with a gradient of 20% of B in A up to 50% over 30 minutes at 4 ml/minute. The peptide is collected manually. After evaporation of the solvents, the purified peptide is lyophilized before being characterized by mass spectrometry and NMR.

### 2—Pseudopeptides

### 2.1 Reduced Peptides

a—Procedure for Preparing Fmoc-leucinal (Douat C., Heitz A., Martinez J., Fehrentz J. A., Tetrahedron Lett., 2000, 41, 37-40): this procedure is summarized by scheme 1 below:

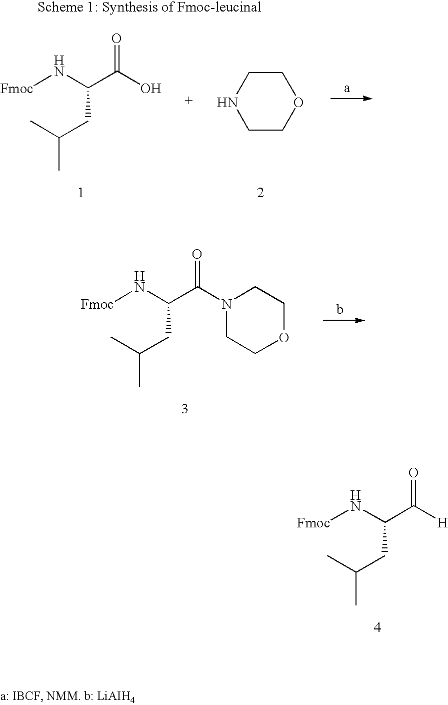

b—Synthesis of Fmoc-Leu-N(CH₂—CH₂)₂O:

Fmoc-Leu-H was synthesized as described by Douat et al. (§a above). 4.81 mmol (0.53 ml) of N-methylmorpholine and 4.81 mmol (0.62 ml) of isobutyl chloroformate (IBCF) are added dropwise, at −15° C., to a solution of Fmoc-Leu-OH (4.81 mmol, 1.7 g) in anhydrous THF (10 ml) under a stream of nitrogen. The solution is stirred with a magnetic bar coupled to a magnetic stirrer plate. The reaction medium is stirred for 15 minutes, filtered and washed twice with anhydrous THF. Still under nitrogen, 4.81 mmol (0.42 ml) of morpholine are added dropwise and the mixture is stirred at ambient temperature for 1 hour. The solvent is evaporated off under vacuum on a rotary evaporator and the residue is taken up with 50 ml of ethyl acetate, and washed with a 5% aqueous KHSO₄ solution (15 ml), a 5% aqueous KHCO₃ solution (15 ml) and then deionized water (2×10 ml). The organic phase is dried over MgSO₄ and evaporated under vacuum on a rotary evaporator. The crude product (1.88 g) is purified by silica column chromatography with elution being carried out with a 70:30 ethyl acetate:hexane mixture (Rf=0.40). The product is in the form of a white foam (69% yield, 1.4 g, 3.31 mmol).

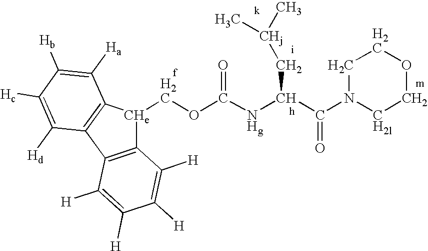

¹H NMR (300 MHz, CDCl₃): 0.94 ppm (3H, d, Jₖ₋ⱼ=6.5 Hz, Hₖ); 0.99 ppm (3H, d, Jₖ₋ⱼ=6.5 Hz, Hₖ); 1.54 ppm (2H, m, Hᵢ); 1.69 ppm (1H, m, Hⱼ); 3.47 ppm (4H, m, Hₗ); 3.66 ppm (4H, m, Hₘ); 4.22 ppm (1H, t, J_{e-f}=6.7 Hz, Hₑ); 4.37 ppm (2H, m, H_{f}); 4.70 ppm (1H, m, Hₕ); 5.57 ppm (1H, d, J_{g-h}=8.8 Hz, H_{g}); 7.31 ppm (2H, m, H_{c}); 7.40 ppm (2H, dd, J_{b-a}=J_{b-c}=7.3 Hz, H_{b}); 7.60 ppm (2H, m, H_{d}) ; 7.76 ppm (2H, d, J_{a-b}=7.3 Hz, Hₐ).

The Weinreb amide thus obtained (1.4 g, 3.31 mmol) is dissolved in 30 ml of anhydrous THF, cooled with an ice bath, and 1.25 equivalents of LiAlH₄ (162.3 mg, 4.14 mmol) are then added in small fractions over a period of 10 minutes. The reaction medium is stirred for 40 minutes at 0° C. and then hydrolyzed with a 5% aqueous KHSO₄ solution (5 ml). The product is extracted with diethyl ether (3×30 ml) and the organic phases are combined, dried over MgSO₄ and evaporated under vacuum so as to give the Fmoc-leucinal (794 mg, 2.35 mmol), which is used without subsequent purification.

c—Synthesis on a Solid Support:

The pseudohexapeptide is synthesized on a semiautomatic synthesizer (CNRS, IBMC, Strasbourg, France) using an Fmoc-Ser(tBu)-Wang PS resin crosslinked with 1% of divinylbenzene (Senn Chemicals, Dielsdorf, Switzerland). The strategy used is a conventional Fmoc/tBu protocol. The peptide chain elongation is carried out using 0.5 gram of resin substituted at 0.5 meq./g by successive coupling of Fmoc-amino acids (0.75 mmol), the side chains of asparagine and of threonine being respectively protected with a trityl group and a tert-butyl group. The coupling catalysts are 2-(1H-benzotriazol-1-yl)-1,1,3,3-tetramethyluronium tetrafluoroborate (TBTU) (0.75 mmol), 1-hydroxybenzotriazole (HOBt) (0.75 mmol) and diisopropylethylamine (DIEA) (2.25 mmol) in dimethylformamide (DMF, 5 ml).

The progress of each step is controlled by means of a calorimetric assay using 2,4,6-trinitrobenzenesulfonic acid for Ser, Gly, Leu, Asn and Thr and using chloranil (tetrachloro-1,4-benzoquinone) for Pro. The N-terminal deprotection of the Fmoc group is carried out with a 20% solution of piperidine in DMF.

d—Synthesis of the Reduced Bond Ψ[CH₂—NH]:

This synthesis is summarized by scheme 2 below:

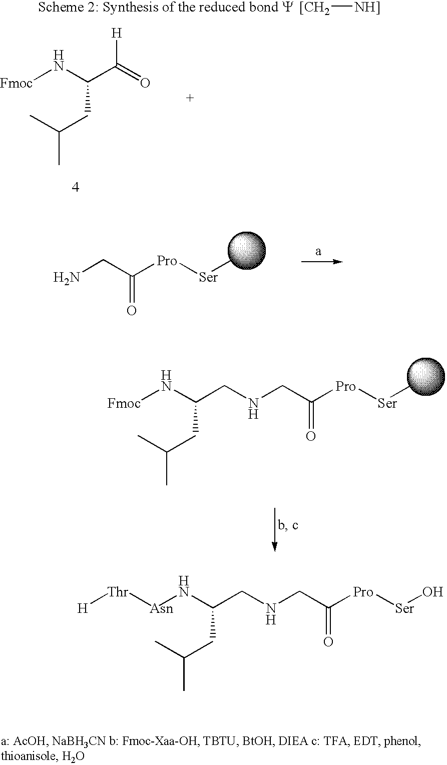

After having successfully coupled Fmoc-Pro-OH and Fmoc-Gly-OH and released the —NH₂ function, the aldehyde Fmoc-Leu-H (0.253 g, 0.75 mmol) is added to the reactor, solubilized in 5 ml of DMF. A few drops of glacial AcOH are added to the reaction medium and 3 eq. of NaBH₃CN are added portionwise over 1 h. The mixture is left overnight with stirring. The Fmoc group is deprotected under the conditions mentioned above.

The synthesis of the hexapseudopeptide is finished by the successive coupling of Fmoc-Asn(Trt)-OH and Fmoc-Thr(tBu)-OH under the conditions mentioned above.

The peptide is cleaved from the resin for 2 hours with a mixture of 10 ml of TFA, 0.750 g of phenol, 0.25 ml of EDT, 0.5 ml of thioanisole and 0.5 ml of deionized water. This mixture is initially cooled to 0° C. but the cleavage is carried out at ambient temperature. The peptide precipitates through the addition of ice-cold Et₂O and the resin is filtered off. The peptide that has remained on the sintered glass is dissolved over a round-bottomed flask full of ice-cold Et₂O using TFA. It is then concentrated and lyophilized.

The pseudopeptide is purified by high performance liquid chromatography (HPLC) carried out on a Hitachi-Merck system equipped with an L6200 pump coupled to a Jasco 875 UV detector. The preparative column used is a Waters DELTA PAK C18 (300×7.8 mm i.d., particle size: 15 μm, porosity: 300 Å) . The eluant is composed of a solution A of 0.1% by volume of TFA (sequencing grade, Sigma) in Ultrapure water and of a solution B of 0.08% of TFA and of 20% of water in acetonitrile (Carlo Erba). The peptide is eluted with a gradient of 20% of B in A up to 50% over 30 minutes at 4 ml/minute. The peptide is collected manually. After evaporation of the solvents, the purified peptide is lyophilized before being characterized by mass spectrometry and NMR.

m/z [ES] theoretical 573.31, experimental 574.41 for [M+H]⁺

The NMR spectrum is in accordance with the expected structure.

### 2.2 Hydrazinopeptides

a—Procedure for the preparation of NβBoc-NβBoc-Nα-Z-Hydrazinoglycine

Boc2N—N(Z)-CH₂—COOH was synthesized according to the method described by N. Brosse et al. (N. Brosse, M.-F. Pinto, J. Bodiguel, B. Jamart-Grégoire J. Org. Chem., 2001, 66, 2869-2873), this synthetic pathway being summarized in scheme 3 below:

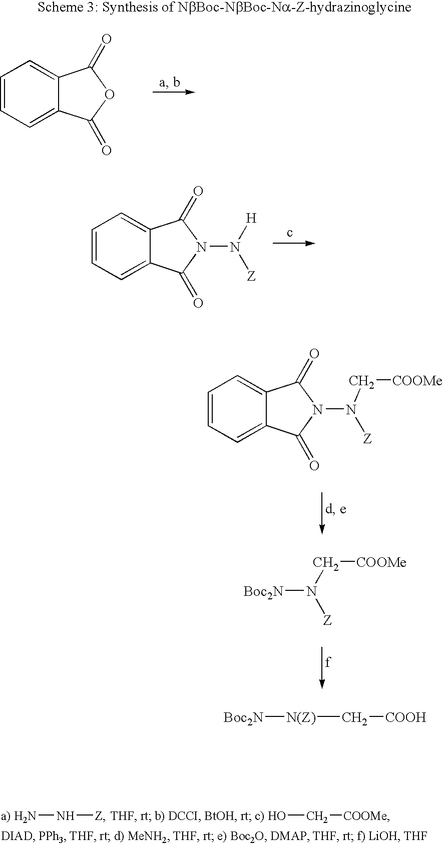

b—Solid-Support Synthesis:

This synthesis is summarized in scheme 4 below.

The pseudohexapeptide is synthesized on a semiautomatic synthesizer (CNRS, IBMC, Strasbourg, France) using an Fmoc-Ser(tBu)-Wang PS resin crosslinked with 1% of divinylbenzene (Senn Chemicals, Dielsdorf, Switzerland). The strategy used is a conventional Boc/Bzl protocol. The peptide chain elongation is carried out using 0.5 gram of resin substituted at 0.69 meq./g by successive coupling of the Boc-amino acids (1.04 mmol), the side chains of asparagine and of threonine being respectively protected with a xanthyl and Bzl group. The Nβ,Nβ-Boc-Nα(Z)Gly-OH is incorporated like a normal amino acid. For this residue, the coupling time is brought to overnight instead of the two hours of reaction for the couplings of the other amino acids. The coupling catalysts are 2-(1H-benzotriazol-1-yl) -1,1,3,3-tetramethyluronium tetrafluoroborate (TBTU) (1.04 mmol), 1-hydroxybenzotriazole (HOBt) (1.04 mmol) and diisopropylethylamine (DIEA) (3.12 mmol) in N,N-dimethylformamide (DMF, 5 ml).

The progression of each step is controlled by means of a calorimetric assay using 2,4,6-trinitrobenzenesulfonic acid for Ser, Gly, Leu, Asn and Thr and chloranil (tetrachloro-1,4-benzoquinone) for Pro. The N-terminal deprotection of the Fmoc group is carried out with a 20% solution of piperidine in DMF.

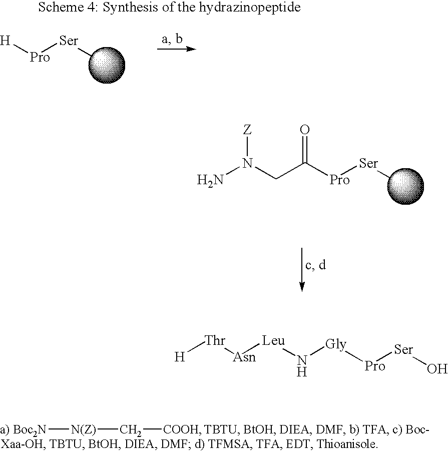

After the coupling of the end threonine, the peptide is cleaved from the resin with a mixture of TFA (10 ml) and TFMSA (1 ml) in the presence of thioanisole (1 ml) and of EDT (0.5 ml). The pseudopeptide is purified by high performance liquid chromatography (HPLC) carried out on a Hitachi-Merck system equipped with an L6200 pump coupled to a Jasco 875 UV detector. The preparative column used is a Waters DELTA PAK C18 (300×7.8 mm i.d., particle size: 15 μm, porosity: 300 Å) . The eluant is composed of a solution A of 0.1% by volume of TFA (sequencing grade, Sigma) in Ultrapure water and of a solution B of 0.08% of TFA and of 20% of water in acetonitrile (Carlo Erba). The peptide is eluted with a gradient of 20% of B in A up to 50% over 30 minutes at 4 ml/minute. The peptide is collected manually. After evaporation of the solvents, the purified peptide is lyophilized before being characterized by mass spectrometry and NMR.

### 2.3 Keto-Methyleneamino Peptides Ψ[CO—CH₂—NH]:

a—Synthesis of Dimethyl Dioxirane (DMD):

254 ml of distilled water, 192 ml of acetone and 58 g of NaHCO₃ are added to a 1 L round-bottomed flask. The mixture is brought to 5° C. and 120 g of Oxone® are added in small portions every 3 min. Each time the oxidant is added, a considerable amount of gas is given off. When the addition is complete, the cold bath is removed and the DMD is recovered by transfer onto a cold wall under a slight vacuum. The solution (≈150 ml at 0.09 M) is conserved on 4 Å molecular sieve at −20° C. and used within 24 h.

b—Oxidation using DMD:

### Synthesis of the Glyoxal Fmoc-Leu-CHO:

Diazo Fmoc-Leu-CH═N₂ (548 mg, 1.5 mmol) is reacted directly by solubilization in the solution of DMD (50 ml, 4.5 mmol). After stirring at 0° C. for 10 min, the solvent is evaporated off and the residue is taken up in DCM (15 ml) in order to remove the residual water through separation by settling out. The solvent is reevaporated and the yield is quantitative. The glyoxal is used without subsequent purification without waiting.

Once the synthesis is complete, the keto-methyleneamino pseudopeptide is cleaved from the resin according to the usual protocol.

This synthetic pathway is summarized in scheme 5 below and is according to Groarke M., Hartzoulakis B., McKervey M. A., Walker B., Williams C. H., Bioorg. Med. Chem. Lett., 2000, 10, 153-155:

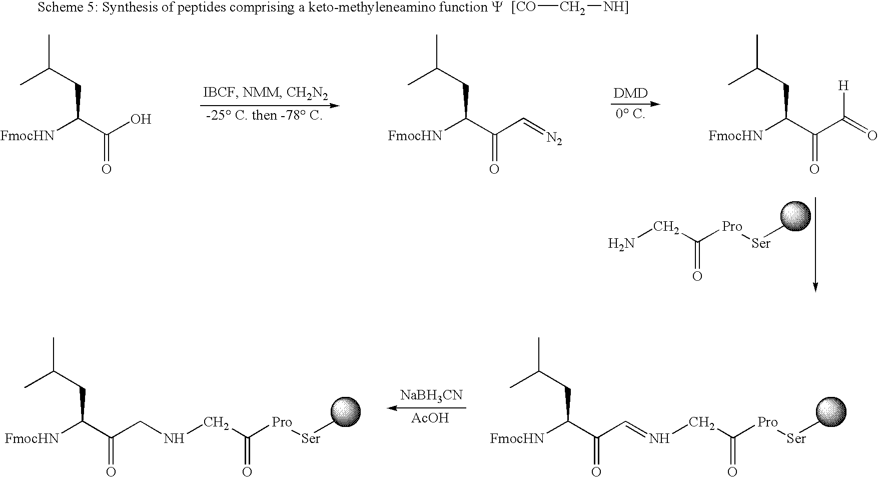

### 2.4 Carbonylhydrazone Peptides Ψ[CO—NH—N=]:

This synthetic pathway is summarized in scheme 6 below and is according to Lourak M., Vanderesse R., Vicherat A., Jamal-Eddine J., Marraud M., Tetrahedron Lett., 2000, 8773-8776:

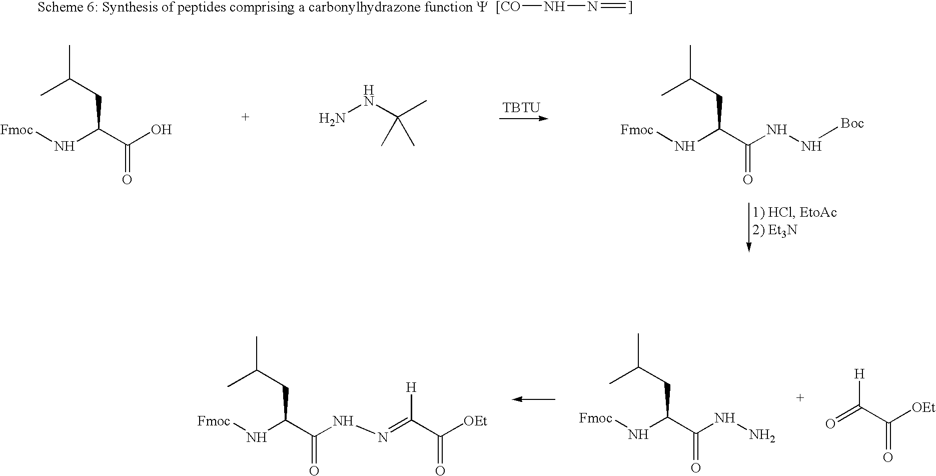

N-Fmoc leucine (1 g, 2.83 mmol) is coupled with tert-butylcarbazate (273 mg, 3.11 mmol) via the formation of an ester activated with TBTU in DCM in the presence of DIEA. The deprotected compound is obtained with a yield of 98%. The Boc protection, which is labile in an acidic medium, is removed by agitation of the compound in a 3N solution of HCl in ethyl acetate for one hour. The hydrazine is then regenerated by the action of a solution of triethylamine (Et₃N) in methanol on the hydrochloride. This reaction is quantitative and clean. The carbonylhydrazone linkage is obtained by condensation of hydrazine on a commercial glycine mimetic, ethyl glyoxylate (1.7 g, 16.64 mmol), as ketone partner. No base is necessary to attain this reaction. A reaction time of 2 hours is sufficient in DCM. The pseudodipeptide diethyl ester is purified on silica gel with an eluent composed of 30% of petroleum ether in ethyl acetate, and recovered in solid form with an 84% yield.

The ester Fmoc-LeuΨ[CO—NH—N=]-Gly-OEt (1.05 g, 2.33 mmol) is solubilized in a ½ (v/v) MeOH/THF mixture at 0° C. 2 equivalents of LiOH (112 mg, 4.66 mmol) are then slowly added and the solute is allowed to stir for 10 min. After evaporation of the mixture of solvents, the residue is taken up in EtOAc and treated by washing with a 5% aqueous KHSO₄ solution (2×10 ml) and distilled water (2×10 ml). After drying over MgSO₄ and evaporation of the solvent, the acid obtained (635 mg, 1.5 mmol) is used, without waiting, in the overnight coupling with the hexapeptide undergoing formation, in the presence of BtOH, TBTU and DIEA, as illustrated by scheme 7.

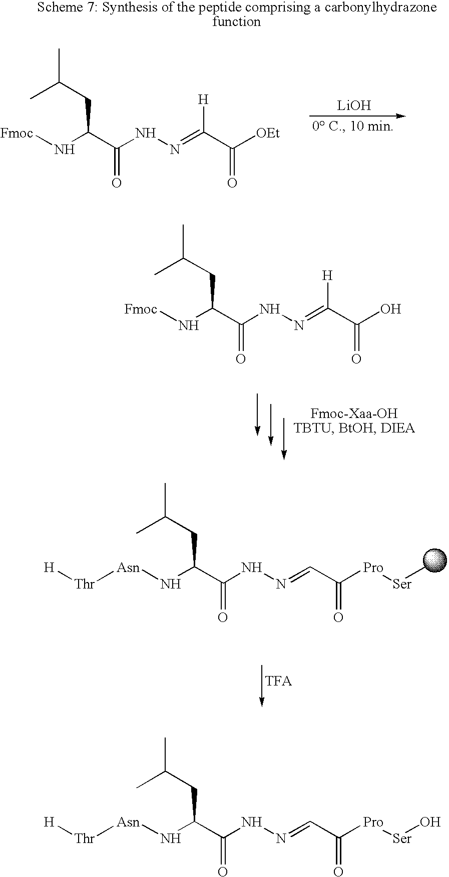

Once the synthesis is complete, the carbonylhydrazone pseudopeptide is cleaved from the resin according to the usual protocol.

### 3. Biotinylated Peptides and/or Peptides Bearing a Para-Benzoylphenylalanine Group

### Synthesis of Biot-Ava-TVT-Bpa-KF:

The Fmoc-Phe-Wang resin (500 mg) is solvated in 5 ml of DMF. After the deprotection step using 3 times 5 ml of 20% piperidine in DMF, Fmoc-Lys(Boc)-OH (513 mg, 3 eq.) dissolved in 5 ml of DMF is added in the presence of TBTU (351 mg, 3 eq.), BtOH (168 mg, 3 eq.) and DIEA (0.6 ml, 9 eq.). After stirring for 40 minutes, a test is carried out on a sample of beads of resin in methanol in the presence of TNBSA. Since the test is negative (observation of a white coloration of the beads), the deprotection step is initiated. Next, Bpa (492.4 mg, 3 eq.) is in turn added, and so on, until the aminovaleric acid Fmoc-Ava-OH is obtained. After deprotection of the Fmoc group, biotin (Bachem, Switzerland) (268 mg, 3 eq.) is finally added, just in the presence of DIEA (0.6 ml, 3 eq.). The stirring is continued overnight. After rinsing of the resin with 5×5 ml of DCM, the resin is dried under vacuum. The peptide and its resin are reacted with a mixture containing 0.75 g of phenol, 0.5 ml of thioanisole, 0.5 ml of osmosed water, 0.25 ml of EDT and 10 ml of TFA. If the addition of the mixture is carried out in an ice bath at 0° C., the stirring is continued for 1 h 30 at ambient temperature. The peptide precipitates with the addition of ice-cold Et₂O and the resin is filtered off. The peptide that has remained on the sintered glass is dissolved over a round-bottomed flask full of ice-cold Et₂O using TFA. It is then concentrated and lyophilized.

The peptides are purified by high performance liquid chromatography (HPLC). The preparative column used is a Waters DELTA PAK C18 (15 μm, 300 Å, 7.8×300 mm). The eluant is composed of a solution A of 0.1% by volume of TFA in water and of a solution B of 0.08% of TFA and of 20% of water in acetonitrile.

### B—Biological Activity

### FIGURES

FIG. 1a represents the evolution of the V0/Vi ratio characteristic of an inhibition involving a single site of the enzyme,

FIG. 1b represents the evolution of the V0/Vi ratio characteristic of a parabolic inhibition in accordance with the reaction scheme represented in FIG. 1c.

### 1. Enzymes

The Xenopus (Xenopus laevis) 26S proteasome was purified according to the protocol described in: GLICKMAN and COUX (2001) Current Protocols in Protein Science, Suppl. 24, Wiley, New York, pp. 21.5.1-21.5.17.

The yeast (Saccharomyces cerevisae) 26S and 20S proteasomes were purified according to the protocol described in: LEGGETT et al. (2002) Molecular Cell, 10, pp 495-507.

### 2. Substrates

The peptidase activities were determined using the fluorogenic substrates Suc-LLVY-amc (CT-L), Z-LLE-βNA (PA) and Boc-LRR-amc (T-L), provided by the company Bachem (Voisins-le-Bretonneux, France).

### 3. Equipment

The enzymatic activities were measured using the BMG Fluostar multiwell plate reader fluorimeter, controlled by Biolise. This apparatus is equipped with a Pelletier-effect thermostating device.

The pH of the buffers was measured using a Radiometer TT1C pH-meter, pH-stat equipped with a B-type electrode.

The mathematical and statistical treatments of the kinetic data were carried out using the Kaleidagraph 3.08.d software (Abelbeck Software).

### 4. Measurement of the Proteasome Activities

The peptidase activities of the yeast and Xenopus 26S proteasomes and those of the yeast 20S proteasome, latent and activated, were determined under the conditions described in Table II.

TABLE II
Conditions for measuring the peptidase
activities of the various enzyme categories.

| | | | Concen- | |
|---|---|---|---|---|
| | | | tration | |
| | | | of the | |
| | | | enzyme | |
| | | Substrate | (μg/ | |
| Proteasome | Activity | (concentration) | ml) | Buffer |
| 26S | CT-L | Suc-LLVY-amc | 1.5 | TrisHCl 20 mm |
| | | (100 μm) | | pH 7.5, |
| | T-L | Boc-LRR-amc | 3 | DTT 1 mm, |
| | | (200 μm) | | MgCl₂ 1 mm |
| | PA | Z-LLE-βNA (200 μm) | 3 | ATP 1 mm, |
| | | | | glycerol 10% |
| 20S latent | CT-L | Suc-LLVY-amc | 30 | TrisHCl 20 mm |
| | | (100 μm) | | pH 7.5, |
| | T-L | Boc-LRR-amc | 60 | DTT 1 mm, |
| | | (200 μm) | | glycerol 10% |
| | PA | Z-LLE-βNA (200 μm) | 60 | |
| 20S | CT-L | Suc-LLVY-amc | 15 | TrisHCl 20 mm |
| activated | | (100 μm) | | pH 7.5, |
| | PA | Z-LLE-βNA (200 μm) | 30 | DTT 1 mm, |
| | | | | glycerol 10%, |
| | | | | SDS 0.02% |

CT-L: chymotrypsin-like activity;
T-L: trypsin-like activity;
PA: post-acid (or caspase) type activity

### 5. Detection and Study of the Inhibitory Effects

The compounds studied are solubilized in the buffer (peptides, pseudopeptides) or in DMSO (lipopeptides, photoactivatable peptides). The enzyme is preincubated (15 min at 30° C.) in the corresponding buffer (Table II), in the presence of the inhibitor. For the cases where the inhibitor is solubilized in DMSO (lipopeptides, photoactivatable peptides), the control without inhibitor contains an amount of DMSO identical to that of the assays with inhibitor (3.5% v/v). The reaction is triggered by adding the substrate. It is continuously monitored for 30 min at 30° C. The initial rates of the assays with inhibitors (calculated from the experimental points) are compared with those of the controls. The results presented were obtained by calculating the mean of at least two independent assays. The variability is less than 10%.

### 5.1—Kinetic Analyses

The IC₅₀ parameter corresponds to the concentration of inhibitor that results in a 50% loss of enzymatic activity.

a. Determination of the IC₅₀ Parameter

The enzyme is preincubated in the presence of increasing concentrations of inhibitor. The reaction is triggered by adding the substrate (see paragraph “Detection and study of the inhibitory effects”). The percentage inhibition is calculated from equation 1.

$$\% inhibition = 100 \times \frac{( {V}_{0} - {V}_{i} )}{{V}_{0}} \qquad eq . 1$$

in which V₀ is the rate of the control, and Vᵢ is the rate in the presence of inhibitor.

The experimental points describe the evolution of the inhibitory effect of the compound studied as a function of its concentration. As a general rule, they fit with the curve described by equation 2 in which [I] is the concentration of inhibitor

$$\% inhibition = \frac{100 \cdot [ I ]}{{IC}_{50} + [ I ]} \qquad eq . 2$$

When the inhibition is cooperative, the experimental points fit with the curve described by equation 3 in which n represents the cooperativity index.

$$\% inhibition = \frac{100 \cdot {[ I ]}^{n}}{{IC}_{50}^{n} + {[ I ]}^{n}} \qquad eq . 3$$

b. Study of the Mechanism of Inhibition

The mechanism of inhibition is determined by tracing the curve of the evolution of the V₀/Vᵢ ratio as a function of the concentration of inhibitor.

### Strict Competitive Inhibition

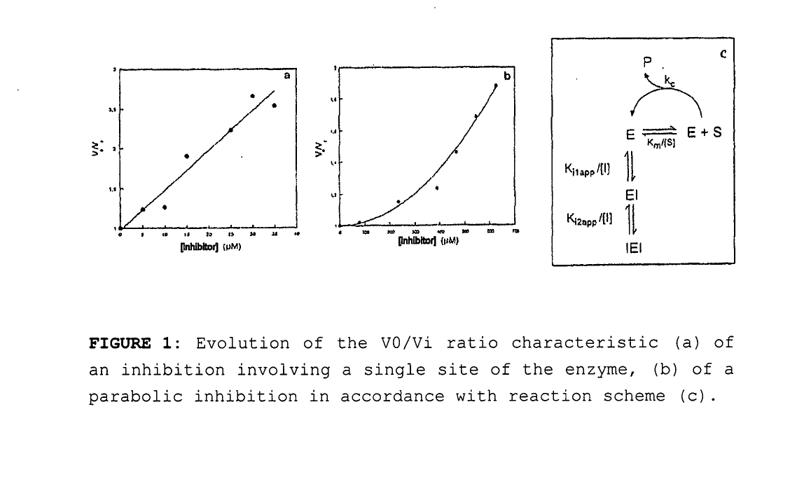

In the case of an inhibition involving a single site of the enzyme, the evolution of the V₀/Vᵢ ratio as a function of the concentration of inhibitor is a straight line (FIG. 1a) defined by equation 4.

$$\frac{{V}_{0}}{{V}_{t}} = 1 + \frac{[ I ]}{{K}_{iapp}} \qquad eq . 4$$

This is the case when the inhibition is strictly competitive: PAPAPOSTOLOU et al., Biochem. Biophys. Res. Comm., 2, 295, 1090-1095 (2002); STEIN et al., Biochemistry, 35, 3899-3908 (1989), with:

$${K}_{iapp} = {K}_{i} + \frac{[ S ]}{{K}_{m}} \qquad eq . 5$$

### Parabolic Inhibition

When the inhibition involves two distinct sites of the enzyme, the evolution of the V₀/Vᵢ ratio as a function of the concentration of inhibitor forms a parabol (FIG. 1b) defined by equation 6, in accordance with the reaction scheme of FIG. 1c.

$$\frac{{V}_{0}}{{V}_{i}} = 1 + \frac{[ I ]}{{K}_{i 1 app}} + \frac{{[ I ]}^{2}}{{K}_{i 1 app} \cdot {K}_{i 2 app}} \qquad eq . 6$$

In the case of the inhibition of the CT-L and PA activities, the first site is a catalytic site, whereas the second would be a noncatalytic regulatory site, the location of which is unknown: PAPAPOSTOLOU et al., Biochem. Biophys. Res. Comm., 2, 295, 1090-1095 (2002); KISSELEV et al., J. Biol. Chem., 278, 35869-35877 (2003).

### 6—Examples

### 6.1 Peptides

By way of comparison, various peptides which are inhibitors of the CT-L activity and of the post-acid activity of the activated 20S proteasome were studied. By way of examples, mention may be made of the peptides TVTFKF (CT-L activity: IC₅₀=229 μM; PA activity: IC₅₀=210 μM) and TITYKF (CT-L activity: IC₅₀=260 μM; PA activity: IC₅₀=336 μM) . They act both on the active sites of the proteasome and on the regulatory sites (parabolic kinetics).

### 6.2 Lipopeptides

Several lipopeptides are inhibitors of the CT-L activity of the activated 20S proteasome.

The inhibitory effect depends on the sequence of the peptide and on the length of the aliphatic chain. A chain CH₃—(CH₂)ₓ—CO— is denoted by CX.

TABLE III
Inhibitory effect of the lipopeptides on the
CT-L activity of the yeast activated 20S proteasome,
after treatment with 35 μM of lipopeptide (17.5 μM for
C18/TVTYKF)

| | C6 | C8 | C10 | C12 | C14 | C16 | C18 |
|---|---|---|---|---|---|---|---|
| TITFDY | 37% | 32% | 35% | 14% | 6% | 20% | 34% |
| TVTYKF | 20% | 50% | 22% | 10% | 0% | | |
| TVTFKF | 32% | 10% | 42% | | | | |

IC₅₀ values of the order of 35 μM are observed for the lipopeptides CH₃—(CH₂)₆—CO-TVTYKF and CH₃—(CH₂)₈—CO-TVTFKF. The C10 carbon chain, when it is attached to the N-terminal end of the peptide TVTFKF, increases the inhibitory capacity by a factor of 6.5 (comparison between CH₃—(CH₂)₈—CO-TVTFKF and the peptide TVTFKF). Similarly, a 17-fold increase is observed by modification of the N-terminal end of TVTYKF with the C8 carbon chain.

For a peptide of given sequence, the inhibitory effect is in general very sensitive to the length of the carbon chain, suggesting that precise modulations of the inhibitory effect may be obtained by simply adjusting this parameter. The lipophilic aliphatic chain is therefore clearly capable of reinforcing the inhibitory effect of the corresponding peptide.

### 6.2 Pseudopeptides

The peptide below was synthesized:

### TNLGPS

The TNLGPS sequence was then used as a starting point for the synthesis of a series of pseudopeptides.

The reduced amide pseudopeptide linkage -ψ[CH₂—NH]- is introduced between the leucine and glycine residues. This bond is nonhydrolyzable.

TNL-ψ[CH₂—NH]-GPS (1)

Ac-TNL-ψ[CH₂—NH]-GPS (2)

The corresponding pseudopeptide TNL-ψ[CH₂—NH]-GPS (1) behaves like an activated 20S proteasome inhibitor. The estimated values of the IC₅₀ for this pseudopeptide is 380 μM, whereas the peptide TLNGPS inhibits the proteasome with an IC₅₀ of 1750 μM (test under experimental conditions where its hydrolysis is negligible). The kinetic analysis shows that pseudopeptide 1 reacts with the catalytic sites and the regulatory site(s).

Pseudopeptide 2 obtained by acetylation of the N-terminal end of 1 is half as effective as 1.

The same order of inhibitory effectiveness is found in relation to the post-acid activity PA: 63% for [1]=500 μM; 28% for [2]=1 mM.

### 6.3 Biotinylated Peptides and/or Peptides Bearing a Para-Benzoylphenylalanine Group

This category is exemplified by the molecule:

Biot-Ava-TVT-Bpa-KF (3) IC₅₀=32 μM

It has a para-benzoylphenylalanine photoactivatable reaction group and a Bpa group (Biot=biotinyl and Ava=δ-aminovaleric acid).

### 7—Proteasome-Activating Effect:

### 7.1 Detection and Quantification of the Activating Effects:

The compounds studied are solubilized in the buffer or in DMSO. The enzyme is preincubated (15 minutes at 30° C.) in the corresponding buffer (Table II), in the presence of the molecule to be tested. When the molecule is solubilized in DMSO, the control (no addition molecule to be tested) contains an amount of DMSO identical to that of the assays(3.5% v/v). The reaction is triggered by adding the substrate. It is continuously monitored for 30 minutes at 30° C. The results presented were obtained by calculating the mean of at least two independent assays. An activation is characterized by an activity, after treatment with the molecule tested, of greater than 100%. The variability is less than 10%. The results are expressed by means of an activation factor fₐ equal to the ratio of the initial rate Vₐ in the presence of the compound tested to the initial rate of the control V₀.

### 7.2 Results:

Several peptides and lipopeptides are activators of the CT-L activity and/or of the T-L activity of the latent 20S proteasome.

| | fₐ | fₐ T-L |
|---|---|---|
| Peptide/lipopeptide | CT-L activity | activity |
| TITFDY | 5 | 3 |
| TVTFKF | 2.3 | 1.7 |
| TITYEY | 2 | — |
| TITYDF | — | 2.5 |
| CH₃—(CH₂)₁₆—CO-TITFDY | 6 | 1.2 |
| CH₃—(CH₂)₁₄—CO-TITFDY | 3 | — |
| CH₃—(CH₂)₁₆—CO-TVTYKF | 3.2 | — |
| CH₃—(CH₂)₁₄—CO-TVTYKF | 2 | — |
| CH₃—(CH₂)₁₂—CO-TVTYKF | 2 | — |

Peptides and lipopeptides therefore constitute molecules that can modulate, with finesse, the CT-L activity by virtue of changes in the aliphatic chain length. The complexity of the effects must be related to the multiplicity of the possible sites of interaction, which are active sites or regulatory sites.

1. A molecule of general formula (I), and the pharmaceutically acceptable salts thereof:
(X₀)ₓ₀—(X₁)ₓ₁—(X₂)ₓ₂—X₃—(X₄)ₓ₄—X₅—X₆—(X₇)ₓ₇—(X₈)ₓ₈—(X₉)ₓ₉ (I)
in which
x₀, x₁, x₂, x₄, x₇, xhd 8 and x₉ each represent, independently, an integer equal to 0 or to 1;
X₀ represents a group chosen from those corresponding to formula (II): 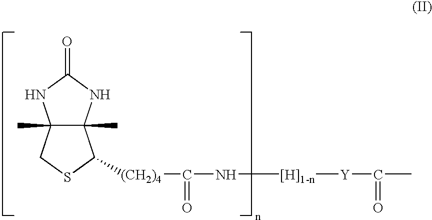
in which Y represents a saturated or unsaturated, linear, branched or cyclic C₁-C₂₄ alkyl group, n represents an integer chosen from 0 and 1;
X₁ and X₃ each represent a natural or synthetic amino acid in the L or D configuration, each comprising at least one hydroxyl function on its side chain;
X₂ represents a natural or synthetic amino acid in the L or D configuration chosen from those comprising an alkyl side chain;
X₄ represents a natural or synthetic amino acid in the L or D configuration which can be chosen from those comprising an aromatic side chain;
X₅ represents an amino acid in the L or D configuration chosen from lysine, arginine, histidine, aspartic acid, asparagine, glutamic acid and glutamine;
X₆ represents an amino acid in the L or D configuration which can be chosen from tyrosine, phenylalanine, leucine, isoleucine, alanine, para-benzoylphenylalanine and lysine;
X₇ represents an amino acid in the L or D configuration which can be chosen from glycine, alanine, leucine, valine, asparagine and arginine;
X₈ represents an amino acid in the L or D configuration which can be chosen from proline, valine, isoleucine and aspartic acid;
X₉ represents an amino acid in the L or D configuration which can be chosen from serine, alanine, lysine, arginine and tryptophan;
the bond between two successive amino acids Xᵢ—Xᵢ₊₁, denoted qᵢ₋ᵢ₊₁, i=1, . . . 8, can be a peptide bond 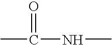
or a pseudopeptide bond chosen from: CO—O, CO—S, CO—CH₂, CO—N(Me), NH—CO, CH═CH, CH₂—CH₂, CH₂—S, CH₂—O, CS—NH, CH₂—NH, CO—CH₂—NH, CO—NH—NH, CO—NH—N═ and CO—N(NH₂);
the amino acids stated above Xᵢ, i=1, . . . 9, being capable of comprising a modification of their α-carbon, denoted Cᵢ, i=1, . . . 9, and bearing the side chain R of the amino acid, which modification consisting of the replacement of: 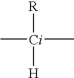
with a group chosen from: 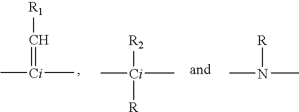
the groups R and CH—R₁ representing the side chain of the amino acid and R₂ representing a C₁-C₆ alkyl group;
R-R₂ can constitute a ring,
the pseudopeptides of the invention also corresponding to the following conditions:
x₀ is equal to 1 or
one of the bonds qᵢ₋ᵢ₊₁, i=1, . . . 8, is a pseudopeptide bond or
one of the Cᵢ, i=1, . . . 9, comprises one of the modifications stated above.

2. A molecule as claimed in claim 1, characterized in that one or more of the following conditions is verified:
at least one of the integers x₀, x₁, X₂, x₄, x₇, x₈ and x₉ is equal to 1;
X₁ and X₃, which may be identical or different, are chosen from threonine and serine;
X₂ is chosen from valine, leucine and isoleucine;
X₄ is chosen from phenylalanine, tryptophan, tyrosine and para-benzoylphenylalanine.

3. A molecule as claimed in claim 1 or claim 2, characterized in that it comprises 4 to 8 amino acids, preferably 5 to 7 amino acids, even more preferably 6 amino acids.

4. A molecule as claimed in any one of claims 1 to 3, characterized in that x₀=1 and the acyl chain —Y—CO— is a linear chain which is represented by the formula —CₚH₂ₚ—CO—, p being an integer ranging from 1 to 23.

5. A molecule as claimed in claim 4, characterized in that:
when n=1, Y represents —CₚH₂ₚ— and p can be 1, 2, 3, 4, 5, 6, 7 or 8;
when n=0, Y represents -CₚH₂ₚ- and p can be an integer ranging from 5 to 23.

6. A molecule as claimed in any one of the preceding claims, characterized in that one or more of the following conditions are verified:
at least one of X₁ and of X₃ represents threonine, preferably X₁ and X₃ both represent threonine,
X₂ is chosen from isoleucine and valine,
X₄ is chosen from phenylalanine, tyrosine and para-benzoylphenylalanine,
at least 2 of the integers x₀, x₁, x₂, x₄, x₇, x₈ and x₉ are equal to 1, even more preferably at least 3 of these integers are equal to 1.

7. A molecule as claimed in claim 1, characterized in that it corresponds to formula (Ia):
X₀—X₁—X₂—X₃—X₄—X₅—X₆ (Ia)
in which the bonds qᵢ, ᵢ₊ₗ between the amino acids Xᵢ and Xᵢ₊₁=1, . . . 5, are peptide or pseudopeptide bonds.

8. A molecule as claimed in claim 7, characterized in that X₀ represents: 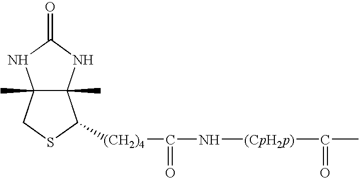
with p ranging from 1 to 8, preferably from 2 to 6, and X₄ represents a para-benzoylphenylalanine group.

9. A molecule as claimed in claim 7, characterized in that X₀ represents a group: 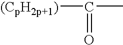
with p ranging from 3 to 23, preferably from 5 to 19.

10. A molecule as claimed in claim 1, characterized in that it corresponds to formula (Ib):
X₃—X₅—X₆—X₇—X₈—X₉ (Ib)
in which:
at least one of the bonds between two successive amino acids is a pseudopeptide bond, or
one of the α-carbons of one of the amino acids is a modified α-carbon.

11. A molecule as claimed in claim 1, characterized in that it belongs to the list:
CH₃(CₙH₂ₙ)—CO-TVTYDY with n=4,6,8,10,12,14,16,18
CH₃(CₙH₂ₙ)—CO-TISYDY with n=4,6,8,10,12,14,16,18
CH₃(CₙH₂ₙ)—CO-TVSYKF with n=4,6,8,10,12,14,16,18
CH₃(CₙH₂ₙ)—CO-TITFDY with n=4,6,8,10,12,14,16,18
CH₃(CₙH₂ₙ)—CO-TITYKF with n=4,6,8,10,12,14,16,18
CH₃(CₙH₂ₙ)—CO-TITYEY with n=4,6,8,10,12,14,16,18
CH₃(CₙH₂ₙ)—CO-TITYDF with n=4,6,8,10,12,14,16,18
CH₃(CₙH₂ₙ)—CO-TVTYKL with n=4,6,8,10,12,14,16,18
CH₃(CₙH₂ₙ)—CO-TVTYKY with n=4,6,8,10,12,14,16,18
CH₃(CₙH₂ₙ)—CO-TVTFKF with n=4,6,8,10,12,14,16,18
CH₃(CₙH₂ₙ)—CO-TITYDL with n=4,6,8,10,12,14,16,18
CH₃(CₙH₂ₙ)—CO-TITFDY with n=4,6,8,10,12,14,16,18
CH₃(CₙH₂ₙ)—CO-TVTFKF with n=4,6,8,10,12,14,16,18
CH₃(CₙH₂ₙ)—CO-TVTYKF with n=4,6,8,10,12,14,16,18
Biot-Ava-TVT-Bpa-KF
Biot-Ava-TVT-Bpa-KY
Biot-Ava-TVT-Bpa-KL
Biot-Ava-TVT-Bpa-DF
Biot-Ava-TVT-Bpa-DY
Biot-Ava-TVT-Bpa-DL
Biot-Ava-TIT-Bpa-KF
Biot-Ava-TIT-Bpa-KY
Biot-Ava-TIT-Bpa-KL
Biot-Ava-TIT-Bpa-DF
Biot-Ava-TIT-Bpa-DY
Biot-Ava-TIT-Bpa-DL
Biot-Ava-TVT-Bpa-EF
Biot-Ava-TVT-Bpa-EY
Biot-Ava-TVT-Bpa-EL
Biot-Ava-TIT-Bpa-EF
Biot-Ava-TIT-Bpa-EY
Biot-Ava-TIT-Bpa-EL
Biot-Ava-TVT-Bpa-NF
Biot-Ava-TVT-Bpa-NY
Biot-Ava-TVT-Bpa-NL
Biot-Ava-TIT-Bpa-NF
Biot-Ava-TIT-Bpa-NY
Biot-Ava-TIT-Bpa-NL
in which Biot represents a biotinyl group, Ava represents a δ-aminovaleric acid group, Bpa represents a para-benzoylphenylalanine group
TNL*GPS
SEK*RVW
TRA*LVR
SNL*NDA
THI*VIK, in which * represents:
a bond chosen from ester, thioester, keto methylene, keto methyleneamino, N-methylamide, inverse amide, Z/E vinylene, ethylene, methylenethio, methyleneoxy, thioamide, methyleneamino, hydrazino, carbonylhydrazone and N-amino bonds, or
the presence of an aza-amino acid as a substitution for one of the amino acids adjacent to *.

12. A molecule, characterized in that it comprises a molecule as claimed in any one of claims 1 to 11 coupled, on its C-terminal end and/or on its N-terminal end, with another molecule which promotes its bioavailability.

13. A medicinal product, characterized in that it comprises a molecule as claimed in any one of claims 1 to 12, in a pharmaceutically acceptable carrier.

14. The use of a molecule as claimed in any one of claims 1 to 12, for preparing a medicinal product for use in the prevention and treatment of a pathology involving the proteasome.

15. The use as claimed in claim 14, characterized in that the pathology is selected from: cancers involving hematological tumors or solid tumors, autoimmune diseases, AIDS, inflammatory diseases, cardiac pathologies and the consequences of ischemic processes whether at the myocardial, cerebral or pulmonary level, allograft rejection, amyotrophy, cerebral strokes, traumas, burns, pathologies associated with aging such as Alzheimer's disease and Parkinson's disease, and the appearance of the signs of aging.

16. The use as claimed in claim 14, for preparing medicinal products for use in the radiosensitization of a tumor.

17. A cosmetic and/or dermatological composition comprising a molecule as claimed in any one of claims 1 to 12, in a cosmetically and/or dermatologically acceptable carrier.

18. A cosmetic process for preventing or treating the appearance of the effects of chronological skin aging and/or of photoaging, characterized in that it comprises the application of a molecule as claimed in any one of claims 1 to 12, in a cosmetically acceptable carrier.


SEQUENCE LISTING (38 sequences)

SEQ ID NO: 1 - protein, 6 aa, Artificial
  Synthetic molecule
XVTYDY

SEQ ID NO: 2 - protein, 6 aa, Artificial
  Synthetic molecule
XISYDY

SEQ ID NO: 3 - protein, 6 aa, Artificial
  Synthetic molecule
XVSYKF

SEQ ID NO: 4 - protein, 6 aa, Artificial
  Synthetic molecule
XITFDY

SEQ ID NO: 5 - protein, 6 aa, Artificial
  Synthetic molecule
XITYKF

SEQ ID NO: 6 - protein, 6 aa, Artificial
  Synthetic molecule
XITYEY

SEQ ID NO: 7 - protein, 6 aa, Artificial
  Synthetic molecule
XITYDF

SEQ ID NO: 8 - protein, 6 aa, Artificial
  Synthetic molecule
XVTYKL

SEQ ID NO: 9 - protein, 6 aa, Artificial
  Synthetic molecule
XVTYKY

SEQ ID NO: 10 - protein, 6 aa, Artificial
  Synthetic molecule
XVTFKF

SEQ ID NO: 11 - protein, 6 aa, Artificial
  Synthetic molecule
XITYDL

SEQ ID NO: 12 - protein, 6 aa, Artificial
  Synthetic molecule
XITFDY

SEQ ID NO: 13 - protein, 6 aa, Artificial
  Synthetic molecule
XVTFKF

SEQ ID NO: 14 - protein, 7 aa, Artificial
  Synthetic molecule
XVTFYKF

SEQ ID NO: 15 - protein, 6 aa, Artificial
  Synthetic molecule
XVTXKF

SEQ ID NO: 16 - protein, 6 aa, Artificial
  Synthetic molecule
XVTXKY

SEQ ID NO: 17 - protein, 6 aa, Artificial
  Synthetic molecule
XVTXKL

SEQ ID NO: 18 - protein, 6 aa, Artificial
  Synthetic molecule
XVTXDF

SEQ ID NO: 19 - protein, 6 aa, Artificial
  Synthetic molecule
XVTXDY

SEQ ID NO: 20 - protein, 6 aa, Artificial
  Synthetic molecule
XVTXDL

SEQ ID NO: 21 - protein, 6 aa, Artificial
  Synthetic molecule
XITXKF

SEQ ID NO: 22 - protein, 6 aa, Artificial
  Synthetic molecule
XITXKY

SEQ ID NO: 23 - protein, 6 aa, Artificial
  Synthetic molecule
XITXKL

SEQ ID NO: 24 - protein, 6 aa, Artificial
  Synthetic molecule
XITXDF

SEQ ID NO: 25 - protein, 6 aa, Artificial
  Synthetic molecule
XITXDY

SEQ ID NO: 26 - protein, 6 aa, Artificial
  Synthetic molecule
XITXDL

SEQ ID NO: 27 - protein, 6 aa, Artificial
  Synthetic molecule
XVTXEF

SEQ ID NO: 28 - protein, 6 aa, Artificial
  Synthetic molecule
XVTXEY

SEQ ID NO: 29 - protein, 6 aa, Artificial
  Synthetic molecule
XVTXEL

SEQ ID NO: 30 - protein, 6 aa, Artificial
  Synthetic molecule
XITXEF

SEQ ID NO: 31 - protein, 6 aa, Artificial
  Synthetic molecule
XITXEY

SEQ ID NO: 32 - protein, 6 aa, Artificial
  Synthetic molecule
XITXEL

SEQ ID NO: 33 - protein, 6 aa, Artificial
  Synthetic molecule
XVTXNF

SEQ ID NO: 34 - protein, 6 aa, Artificial
  Synthetic molecule
XVTXNY

SEQ ID NO: 35 - protein, 6 aa, Artificial
  Synthetic molecule
XVTXNL

SEQ ID NO: 36 - protein, 6 aa, Artificial
  Synthetic molecule
XITXNF

SEQ ID NO: 37 - protein, 6 aa, Artificial
  Synthetic molecule
XITXNY

SEQ ID NO: 38 - protein, 6 aa, Artificial
  Synthetic molecule
XITXNL